(19) United States
(12) Patent Application Publication
(10) Pub. No.: US 2011/0077293 A1
(43) Pub. Date: Mar. 31, 2011

(54) GAMBOGIC AMINE, A SELECTIVE TrkA AGONIST WITH NEUROPROTECTIVE ACTIVITY

(75) Inventor: Keqiang Ye, Atlanta, GA (US)

(73) Assignee: EMORY UNIVERSITY, Atlanta, GA (US)

(21) Appl. No.: 12/676,964

(22) PCT Filed: Sep. 10, 2008
(86) PCT No.: PCT/US08/75859

§ 371 Date: Nov. 23, 2010

Related U.S. Application Data
(60) Provisional application No. 60/993,763, filed on Sep. 14, 2007.

(51) Int. Cl. A61K 31/35; C07D 311/78; A61P 25/00

(52) U.S. Cl. 514/453; 549/382

(57) ABSTRACT

Small molecule agonists, partial agonists, and antagonists for the TrkA receptor are described. The compounds are gambogic amines, where the carboxylic acid group of gambogic acid (CO₂H) has been replaced by an amine group (CH₂NR₁R₂). In some embodiments, the compounds selectively bind to TrkA but not TrkB or C, robustly induce its tyrosine phosphorylation and downstream signaling activation including Akt and MAP kinases. Further, they can strongly prevent glutamate-induced neuronal cell death and provoke prominent neurite outgrowth in PC12 cells. Gambogic amines specifically interact with the cytoplasmic juxtamembrane domain of TrkA receptor and trigger its dimerization. Administration of these compounds in can substantially diminishes Kainic acid-triggered neuronal cell death and decrease infarct volume in transient middle cerebral artery occlusion (MCAO) model of stroke. Thus, these compounds can provide effective treatments for debilitating neurodegenerative diseases and provide neuroprotection from patients suffering from stroke or other ischemic events.

### CROSS-REFERENCE TO RELATED APPLICATIONS

This application claims the benefit under 35 USC 119 of U.S. Provisional Patent Application No. 60/993,763 filed Sep. 14, 2007. The disclosure of said U.S. Provisional Patent Application No. 60/993,763 is hereby incorporated herein by reference, in its respective entirety, for all purposes.

### FIELD OF THE INVENTION

The invention is generally in the area of selective TrkA agonists, in particular, Gambogic amines, and methods of using the compounds to provide neuroprotection and/or treat or prevent neurodegenerative disorders.

### BACKGROUND OF THE INVENTION

Neurotrophins play an essential role in the development and maintenance of the peripheral and the central nervous systems. The receptors for neurotrophins are members of a family of highly similar transmembrane tyrosine kinases (TrkA, TrkB and TrkC). Each neurotrophin binds to a preferred receptor in the family: nerve growth factor (NGF) binds mainly TrkA, brain-derived neurotrophic factor (BDNF) and neurotrophin-4 (NT-4) bind TrkB and neurotrophin-3 binds TrkC, whereas p75NTR receptor non-selectively interacts with all members of the neurotrophins with similar affinity. Docking of TrkA by NGF initiates receptor dimerization, catalytic phosphorylation of cytoplasmic tyrosine residues on the receptor, and a cascade of cell-signaling events including activation of PI 3-kinase/Akt, Ras/Raf/MAP kinase and PLC-γ1 signaling pathways (Kaplan and Stephens, 1994). These signals lead to prevention of apoptotic cell death, promotion of cellular differentiation and axon elongation, and up-regulation of choline acetyl transferase (ChAT). Several neuronal cell types including sensory, sympathetic, and cholinergic neurons, implicated in various diseases, express TrkA and therefore respond to NGF (Mufson et al., 1997). In the normal adult human central nervous system (CNS), the cortical and nucleus basalis neurons that comprise the cholinergic system play essential roles in learning and memory.

In neurodegenerative processes, such as mild cognitive impairment (MCI), loss of TrkA density correlates with neuronal atrophy and precedes neuronal death and severe cognitive impairment in Alzheimer's Disease (AD) (Counts et al., 2004). In MCI-AD progression, loss of TrkA correlates with cognitive decline. In basal forebrain neurons of aged rats, the expression of NGF receptors is decreased, but can be reversed by NGF administration (Backman et al., 1997). It has been suggested that NGF therapy may delay the onset of Alzheimer's disease (Barinaga, 1994; Lindsay, 1996) and ameliorate peripheral diabetic neuropathies (Ebadi et al., 1997). Other applications proposed for NGF include treatment of neuronal damage (Hughes et al., 1997) and targeting of neuroectoderm-derived tumors (Cortazzo et al., 1996; LeSauteur et al., 1995). Both in vitro and in animal models studies demonstrate that NGF might be clinically useful for treating CNS diseases (Connor and Dragunow, 1998). As expected, preclinical and clinical findings also suggest that subcutaneous or intravenous administration of neurotrophins may be an effective treatment for peripheral neurodegenerative disorders (McArthur et al., 2000; McMahon and Priestley, 1995).

Despite the therapeutic potential of NGF, clinical trials featuring this protein have been disappointing (Verrall, 1994). There are several reasons for this, including the inherent drawbacks associated with using polypeptides as drugs (Saragovi et al., 1992), in vivo instability (Barinaga, 1994), and pleiotrophic effects due to activation of signals that were not intentionally targeted (e.g., those mediated via the low-affinity NGF receptor p75) (Carter and Lewin, 1997). Moreover, NGF protein is relatively expensive to produce for medicinal applications. In order to circumvent the above curbs, substantial efforts have been made to design small, proteolytically stable molecules with neurotrophic activity, selective for cells expressing TrkA (Lee and Chao, 2001; Saragovi et al., 1991; Saragovi et al., 1992). They present partial agonistic activity in the absence of NGF by activating TrkA. Although they do not directly dimerize the receptor, the compounds cause conformational changes on TrkA that stabilize its dimerization. Thus, they act more as potentiators of NGF action rather than as robust TrkA agonists.

It would be advantageous to provide selective TrkA agonists, and methods of using these compounds. The present invention provides such compounds and methods.

### SUMMARY OF THE INVENTION

Gambogic amines are disclosed. Depending on the side chains on the amine group, the compounds may be TrkA agonists, partial agonists, or antagonists. The compounds bind with relatively high affinity to TrkA. Those compounds which cause apoptosis can be used as anti-cancer agents, and those which inhibit apoptosis (in which case they may be selective TrkA agonists) can be used as neuroprotective compounds. Assays for determining the activity of the compounds are described herein.

Certain Gambogic amines selectively bind to TrkA, trigger its tyrosine phosphorylation, elicit PI 3-kinase/Akt and MAP Kinase activation, provoke neurite outgrowth in PC12 cells, and/or prevent neuronal cell death. Moreover, they can substantially decrease the infarct volume following MCAO. Thus, these gambogic amines can mimic NGF and possess potent neurotrophic activities.

The selective TrkA agonists can be used to provide neuroprotection, before, during, or after a neural insult, such as a stroke or other ischemic event. The compounds can also be used to treat demyelinating diseases such as multiple sclerosis (MS), to enhance nerve regeneration, and/or to treat or prevent neurodegenerative diseases. Such neurodegenerative diseases include, but are not limited to, epilepsy, head and spinal chord trauma, Parkinson disease, Huntington's disease, Alzheimer's disease, or amyotrophic lateral schlerosis, or a neurological disorder.

The selective TrkA agonists or partial agonists, and methods of providing neuroprotection, or treating and/or preventing neurodegenerative diseases or demyelinating diseases, will be better understood with reference to the detailed description below.

### DETAILED DESCRIPTION

Gambogic amines, pharmaceutical compositions including the gambogic amines, methods of preparing the gambogic amines, and methods of treatment and prevention using the gambogic amines, are disclosed. The present invention arises out of the discovery that gambogic amines are selective TrkA modulators (i.e., agonists, partial agonists, or antagonists). Therefore, these compounds are potentially useful for providing neuroprotection, treating demyelinating disorders, and for treating and/or preventing neurodegenerative disorders.

### I. Gambogic Amines

The gambogic amines themselves generally have the following formula.

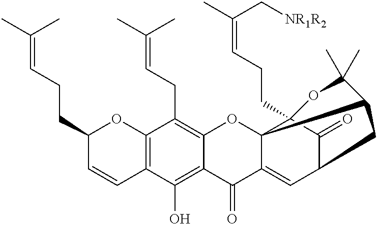

The gambogic amines can be prepared, for example, using Scheme 1 as shown below. Where gambogic acid is used as a precursor, it can be purified by 1) preparation of the pyridine salt of the crude extract from gamboge (resin from Garcinia hanburyi Hook) followed by repeated recrystallization of the salt in ethanol or 2) converting the salt to the free acid. Using this procedure, about 10% by weight of gambogic acid with purity >99% (HPLC) can be obtained from the crude extract. Where gambogic amide is used as a precursor (and the amide is reduced to the amine), it can be obtained, for example, by converting the gambogic acid to the amide using known amidation chemistry.

The synthesis of gambogic amines is outlined in the following reaction scheme, Scheme 1.

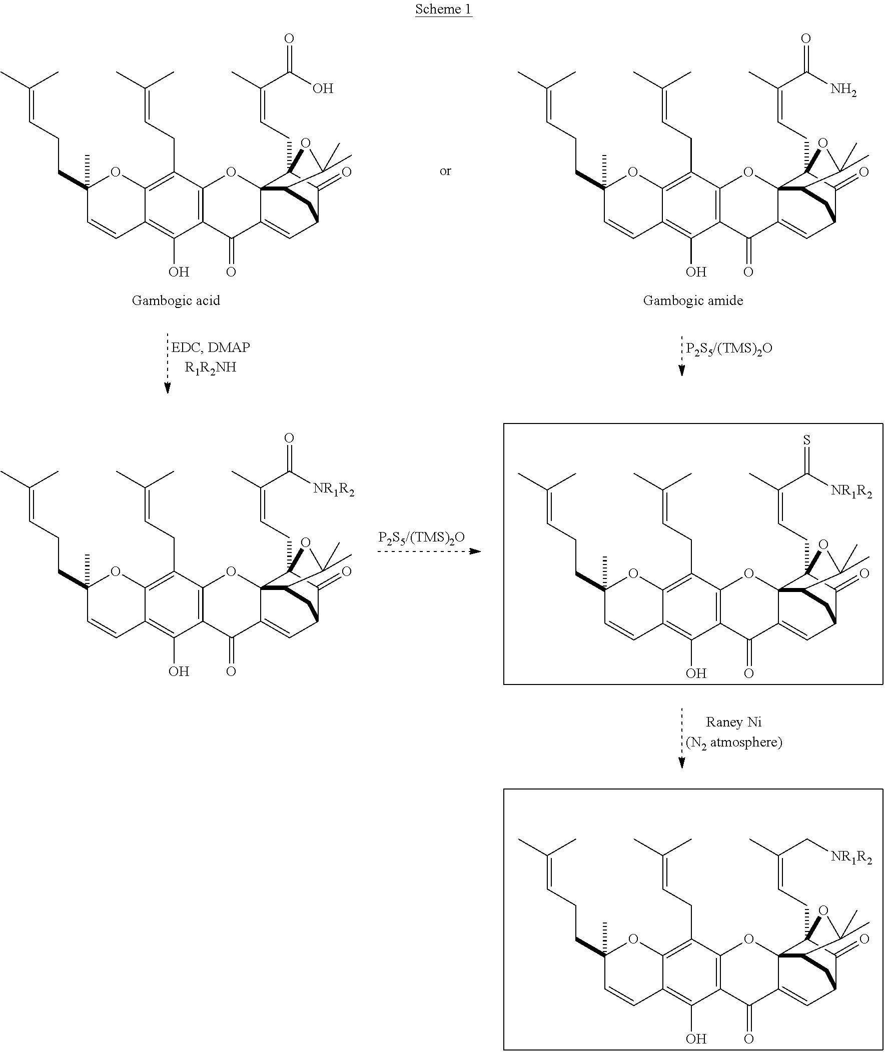

As shown in Scheme 1, Gambogic amine derivatives can be formed at least using either one of two pathways: 1) a three-step protocol involving diimide coupling of gambogic acid with various primary and secondary amines to form gambogic amide derivatives, followed by thioamide formation, and finally, thiocarbonyl reduction by nickel under an N₂ atmosphere; or 2) direct thioamide formation from gambogic amide followed by thiocarbonyl reduction under nitrogen to give the free primary amine The R1 and R2 groups can be any size alkyl group or carbonyl groups. A variety of gambogic amine derivatives can be prepared by using different R1 and R2 groups. Alternatively, starting with the unsubstituted compound (R1 and R2 are H), one can use alkyl halides, alkylaryl halides (such as benzyl halides), or other such alkylating agents to place suitable substituents (including those with the substituents described below) on the nitrogen. Such reactions are well known to those of skill in the art.

Gambogic amide exhibits potent neurotrophic effects in protecting hippocampal neurons from OGD (Oxygen and Glucose Deprivation) or glutatmate-triggered neuronal cell death in vitro. In addition, gambogic amide can strongly block kainic acid-elicited neuronal cell death in mouse brain. Further, gambogic amide can also effectively decrease the stroke-induced infarct volume. A structure-activity assay was performed, which revealed that the carboxy group in gambogic acid (and, hence, gambogic amide) is not essential for gambogic acid family members' neurotrophic effects. Hence, the gambogic amine compounds described herein were developed, to maintain the activity of the gambogic amide, while improving the water-solubility of the compounds.

Gambogic amine derivatives can further improve the biological effect of gambogic amide. The gambogic amine derivatives are more water soluble than gambogic amide, and in some embodiments, exhibit even stronger neurotrophic activity in suppressing neuronal cell death, leading to better therapeutic efficacy in treating neurodegenerative diseases such as Alzheimer's disease.

The compounds have certain effects on apoptosis in cancer cells, and are believed to modulate (i.e., to agonize, partially agonize, or antagonize) the TrkA receptor, depending on the particular compound structure. The assays described herein can be used in a high-throughput manner to identify which compounds within this family of compounds has desired activity. Indeed, compounds with pronounced apoptotic activity may have considerable efficacy as anti-cancer drugs, whereas compounds with enhanced protective activity may have considerable efficacy for providing neuroprotection, treating neurodegenerative disorders, and the like.

There are many functional groups in the structure of the gambogic amines which can be modified. These include, but are not limited to, the hydroxy group, which may be converted to an ether, ester or other functional groups; the carbon-carbon double bond between C-9 and C-10 is part of an α,β-unsaturated ketone, which can react with a nucleophile, be reduced to a carbon-carbon single bond, or may be converted to an epoxide, which in turn may undergo further reaction; the carbon-carbon double bond between C-27 and C-28 is part of an α,β-unsaturated carboxyl, that may also react with a nucleophile, be reduced to a carbon-carbon single bond, or may be converted to a cyclopropane ring, which in turn may undergo further reaction; the two isoprene carbon-carbon double bonds at C-37/C-38 and C-32/C-33, may also be reduced to a carbon-carbon single bond, be cleaved to form an aldehyde group or a carboxyl group, both of which may be modified to other functional groups, or be converted to an epoxide, which in turn may undergo further reaction; the carbon-carbon double bond between C-3 and C-4 may also be reduced to a carbon-carbon single bond, or be converted to an epoxide that may undergo further reaction; the ketone group at C-12 may be reduced to an alcohol, or may be converted to an oxime, a semicarbazone, or an amino group; the other ketone group may also be reduced, or may be converted to other functional groups. In short, many derivatives of the gambogic amines shown above can be prepared, and such derivatives are intended to be within the scope of the invention.

Optional substituents on the alkyl, aryl, and arylalkyl groups R1 and R2 include one or more halo, hydroxy, carboxyl, alkoxycarbonyl, amino, nitro, cyano, C₁-C₆ acylamino, C₁-C₆ aminoacyl, C₁-C₆ acyloxy, C₁-C₆ alkoxy, aryloxy, alkylthio, C₆-C₁₀ aryl, C_{d}-C₇ cycloalkyl, C₂-C₆ alkenyl, C₂-C₆ alkynyl, C₆-C₁₀ aryl(C₂-C₆)alkenyl, C₆-C₁₀ aryl(C₂-C₆)alkynyl, saturated or partially saturated 5-7 membered heterocyclo group, or heteroaryl.

Useful alkyl groups include straight-chained and branched C₁₋₁₀ alkyl groups, more preferably C₁₋₆ alkyl groups. Typical C₁₋₁₀ alkyl groups include methyl, ethyl, propyl, isopropyl, butyl, sec-butyl, tert-butyl, 3-pentyl, hexyl and octyl groups, which may be optionally substituted.

Useful alkoxy groups include oxygen substituted by one of the C₁₋₁₀ alkyl groups mentioned above, which may be optionally substituted.

Useful alkylthio groups include sulphur substituted by one of the C₁₋₁₀ alkyl groups mentioned above, which may be optionally substituted. Also included are the sulfoxides and sulfones of such alkylthio groups.

Useful amino groups include —NH₂, —NHR₁, and —NR₁R₂, wherein R₁ and R₂ are C₁₋₁₀ alkyl or cycloalkyl groups, or R₁ and R₂ are combined with the N to form a ring structure, such as a piperidine, or R₁ and R₂ are combined with the N and another heteroatom to form an optionally substituted, saturated or partially saturated 5-7 membered heterocyclo group, such as a piperazine. The alkyl group may be optionally substituted.

Useful heteroatoms include N, O or S.

Optional substituents on the aryl, aralkyl and heteroaryl groups include one or more acyl, alkylenedioxy (—OCH₂O—), halo, C₁-C₆ haloalkyl, C₆-C₁₀ aryl, C₆-C₇ cycloalkyl, C₁-C₆ alkyl, C₂-C₆ alkenyl, C₂-C₆ alkynyl, C₆-C₁₀ aryl(C₁-C₆)alkyl, C₆-C₁₀ aryl(C₂-C₆)alkenyl, C₆-C₁₀ aryl(C₂-C₆)alkynyl, C₁-C₆ hydroxyalkyl, nitro amino, ureido, cyano, C₁-C₆ acylamino, hydroxy, thiol, C₁-C₆ acyloxy, azido, C₁-C₆ alkoxy, or carboxy.

Useful heteroalkyl groups contain 1-10 carbon atoms and 1, 2 or 3 heteroatoms. Examples of heteroalkyl groups include —CH₂CH₂OCH₂CH₃, —CH₂CH₂OCH₂CH₂OCH₂CH₃, —CH₂CH₂NHCH₃, —CH₂CH₂N(CH₂CH₃)₂, —CH₂ CH₂OCH₂CH₂NHCH₃, —CH₂CH₂OCH₂CH₂OCH₂CH₂NHCH₃, —CH₂ CH₂ NHCH₂CH₃, —CH₂C(CH₃)₂CH₂N(CH₃)₂ or —CH₂(N-ethylpyrrolidine), which may be optionally substituted.

Optional substituents on heteroalkyl groups include one or more halo, hydroxy, carboxyl, amino, nitro, cyano, alkyl, C₁-C₆ acylamino, C₁-C₆ aminoacyl, C₁-C₆ acyloxy, C₁-C₆ alkoxy, aryloxy, alkylthio, C₆-C₁₀ aryl, C_{d}-C₇ cycloalkyl, C₂-C₆ alkenyl, alkenoxy, C₂-C₆ alkynyl, C₆-C₁₀ aryl(C₂-C₆)alkenyl, C₆-C₁₀ aryl(C₂-C₆)alkynyl, saturated and unsaturated heterocyclic, or heteroaryl.

Useful aryl groups are C₆₋₁₄ aryl, especially C₆₋₁₀ aryl. Typical C₆₋₁₄ aryl groups include phenyl, naphthyl, phenanthrenyl, anthracenyl, indenyl, azulenyl, biphenyl, biphenylenyl and fluorenyl groups.

Useful cycloalkyl groups are C₃₋₈ cycloalkyl. Typical cycloalkyl groups include cyclopropyl, cyclobutyl, cyclopentyl, cyclohexyl and cycloheptyl.

Useful saturated or partially saturated carbocyclic groups are cycloalkyl groups as defined above, as well as cycloalkenyl groups, such as cyclopentenyl, cycloheptenyl and cyclooctenyl.

Useful halo or halogen groups include fluorine, chlorine, bromine and iodine.

Useful aralkyl groups include any of the above-mentioned C₁₋₁₀ alkyl groups substituted by any of the above-mentioned C₆₋₁₄ aryl groups. Useful values include benzyl, phenethyl and naphthylmethyl.

Useful haloalkyl groups include C₁₋₁₀ alkyl groups substituted by one or more fluorine, chlorine, bromine or iodine atoms, e.g. fluoromethyl, difluoromethyl, trifluoromethyl, pentafluoroethyl, 1,1-difluoroethyl, chloromethyl, chlorofluoromethyl and trichloromethyl groups.

Useful acylamino groups are any C₁₋₆ acyl(alkanoyl) attached to an amino nitrogen, e.g. acetamido, propionamido, butanoylamido, pentanoylamido, hexanoylamido as well as aryl-substituted C₂₋₆ substituted acyl groups.

Useful acyloxy groups are any C₁₋₆ acyl(alkanoyl) attached to an oxy (—O—) group, e.g. formyloxy, acetoxy, propionoyloxy, butanoyloxy, pentanoyloxy, hexanoyloxy and the like.

Useful saturated or partially saturated 5-7 membered heterocyclo groups include tetrahydrofuranyl, pyranyl, piperidinyl, piperazinyl, pyrrolidinyl, imidazolidinyl, imidazolinyl, indolinyl, isoindolinyl, quinuclidinyl, morpholinyl, isochromanyl, chromanyl, pyrazolidinyl pyrazolinyl, tetronoyl and tetramoyl groups.

Optional substitutents on the 5-7 membered heterocyclo groups include one or more heteroaryl, heterocyclo, alkyl, aralkyl, cycloalkyl, alkoxycarbonyl, carbamyl, aryl or C₁-C₆ aminoacyl.

Useful heteroaryl groups include any one of the following: thienyl, benzo[b]thienyl, naphtho[2,3-b]thienyl, thianthrenyl, furanyl, pyranyl, isobenzofuranyl, chromenyl, xanthenyl, phenoxanthiinyl, 2H-pyrrolyl, pyrrolyl, imidazolyl, pyrazolyl, pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalzinyl, naphthyridinyl, quinozalinyl, cinnolinyl, pteridinyl, carbazolyl, _{b}eta.-carbolinyl, phenanthridinyl, acrindinyl, perimidinyl, phenanthrolinyl, phenazinyl, isothiazolyl, phenothiazinyl, isoxazolyl, furazanyl, phenoxazinyl, 1,4-dihydroquinoxaline-2,3-dione, 7-aminoisocoumarin, pyrido[1,2-a]pyrimidin-4-one, 1,2-benzoisoxazol-3-yl, benzimidazolyl, 2-oxindolyl and 2-oxobenzimidazolyl. Where the heteroaryl group contains a nitrogen atom in a ring, such nitrogen atom may be in the form of an N-oxide, e.g. a pyridyl N-oxide, pyrazinyl N-oxide, pyrimidinyl N-oxide and the like.

Optional substituents on the heteroaryl groups include one or more heteroaryl, heterocyclo, alkyl, aralkyl, cycloalkyl, alkoxycarbonyl, carbamyl, aryl and C₁-C₆ aminoacyl.

Certain compounds may exist as stereoisomers including optical isomers. The invention includes all stereoisomers and both the racemic mixtures of such stereoisomers as well as the individual enantiomers that may be separated according to methods that are well known to those of ordinary skill in the art.

Examples of pharmaceutically acceptable addition salts include inorganic and organic acid addition salts such as hydrochloride, hydrobromide, phosphate, sulphate, citrate, lactate, tartrate, maleate, fumarate, mandelate and oxalate; and inorganic and organic base addition salts with bases such as sodium hydroxy, Tris(hydroxymethyl)aminomethane (TRIS, tromethane) and N-methyl-glucamine

Examples of prodrugs include the simple amides of the gambogic amines with various carboxylic acid-containing compounds; imines of the amino groups (e.g. those obtained by condensation with a C₁₋₄ aldehyde or ketone according to methods known in the art); and carbamates of the amine groups.

### II. High Throughput Screening Method

The following high-throughput screening assay was developed to identify small molecules that mimic NGF and activate TrkA, ideally in a selective manner. That is, the assay can identify compounds that are agonists or partial agonists for TrkA, and ideally those which are selective for TrkA over TrkB and TrkC. The gambogic amines described herein can be evaluated using this method for their ability to function as agonists, partial agonists, or antagonists at the TrkA receptor, as well as their selectivity for this receptor over the TrkB and TrkC receptors.

The cell-based apoptotic assay uses a caspase assay system, which include fluorometric, colorimetric, cell proliferation and cell viability assay systems, which include a fluorometric or colorimetric marker for apoptotic cells. In one embodiment, the cell-based apoptotic assay uses a cell permeable fluorescent dye, such as MR(DERD)2, which turns red upon caspase-3 cleavage in apoptotic cells. The cells used in the assay can be from any cell line that does not express TrkA. In one embodiment, the cells are from a murine cell line T17, which was derived from basal forebrain SN56 cells. T17 cells are TrkA stably transfected SN56 cells.

Those candidate compounds that selectively protect T17, but not SN56 cells, are either TrkA agonists, or function by activating a downstream pathway. Even if they are TrkA agonists, it is not clear from the anti-apoptotic activity alone that they are selective TrkA agonists. So, additional screening steps can be performed to determine whether the compounds are TrkA agonists, and if so, whether they are selective TrkA agonists.

To help ascertain whether the compounds act on TrkA, or on a downstream step, one can look at whether the compounds cause TrkA to dimerize. That is, compounds which cause TrkA to dimerize act on TrkA, not on a downstream step. One way to determine whether test compounds can trigger TrkA dimerization is to co-transfect GFP-TrkA and HA-TrkA into HEK293 cells, expose the co-transfected cells to the compounds.

As used herein, a TrkA agonist is a compound with at least 90% of the activity of NGF in selectively protecting T17, but not SN56 cells, and which causes TrkA to dimerize, and a partial agonist is a compound with less than 90% of the activity of NGF in the selective protection of these cells, and which causes TrkA to dimerize. A selective TrkA agonist (or partial agonist) is a compound which exhibits substantial binding affinity to TrkA over TrkB and/or TrkC, for example, at least 4:1 binding affinity for TrkA over TrkB and/or TrkC.

Those compounds which inhibit apoptosis and cause TrkA to dimerize are TrkA agonists (or partial agonists). To determine whether the compounds are selective TrkA agonists (i.e., bind selectively to TrkA in preference to TrkB or TrkC), one can use cells that are cotransfected with TrkB and TrkA receptors or TrkC and TrkA receptors. If the compounds are selective for TrkA, the receptors will fail to dimerize. Selective TrkA agonists will also elicit tyrosine phosphorylation in TrkA, but not in TrkB or C receptors (or in cells co-transfected to express two or more of these receptors, i.e., TrkA in combination with TrkB and/or TrkC). So, one can alternatively, or also, look for tyrosine phosphorylation to determine whether the compounds are selective TrkA agonists (or partial agonists). Dose studies can be performed to determine whether the compounds are agonists or partial agonists.

Once compounds with desired properties are identified, their actual activity can be assessed, for example, by determining whether the compounds provoke neurite outgrowth in PC12 cells, and/or prevent neuronal cell death. Compounds with one or both of these activities can be useful compounds for treating neurodegenerative disorders and/or providing neuroprotection.

Ideally, compounds testing positive in both of these screens can be analyzed for TrkA tyrosine phosphorylation, and/or Akt and MAP kinases signaling cascade activation. Each of the assay steps is described in more detail below.

T17 cells can be cultured in multi-well plates, such as 96-well plates, and preincubated with around 10 μM of putative TrkA agonists for a period of time, for example, 30 min, followed by 1 μM staurosporine (STS) treatment for a period of time, for example, 9 h. When the dye is MR(DEVD)2, and this dye is introduced to the cells (for example, 1 h before examination under a fluorescent microscope), apoptotic cells will appear red, while live cells will have no signal. NGF can be used as a positive control. NGF will substantially decrease the red cell numbers compared to a control, such as a DMSO control.

As discussed below in the Examples section, using the assay using the caspase-3-activated fluorescent dye, biologically active compounds from the Spectrum Collection Library can be screened. When the screen was performed against various gambogic acid derivatives (data not shown), it identified compounds which selectively protected T17, but not SN56 cells, from STS-initiated apoptosis. This indicated that the compounds might act either directly through the TrkA receptor, or its downstream signaling effectors. The analogous screening assay can be used to evaluate the gambogic amines described herein.

Even in the absence of NGF, T17 exhibits a stronger anti-apoptotic effect than its parental SN56 cells, indicating that overexpression of TrkA suppresses caspase-3 activation. NGF treatment further enhances this effect.

Identification of Compounds as Survival Enhancers

To compare the apoptosis inhibitory activity of the compounds, they can be pre-incubated (0.5 μM) with T17 and SN56 cells, followed by 1 μM STS for 9 h. Quantitative analysis of the apoptosis inhibitory activities will reveal which compounds have little or no ability to protect SN56 cells from apoptosis, but which strongly suppress apoptosis in T17 cells ideally with protective activities even stronger than NGF). Such compounds may show use in neuroprotection.

Compounds identified in the screen that increase apoptosis in T17 cells can trigger programmed cell death, and may be useful as anti-cancer agents.

TrkA is highly expressed in hippocampal neurons (Culmsee et al., 2002; Kume et al., 2000; Zhang et al., 1993). TrkA and p75NTR are up-regulated in hippocampal and cortical neurons under pathophysiological conditions (Kokaia et al., 1998; Lee et al., 1998). Moreover, neuroprotective effects of NGF in hippocampal and cortical neurons have been demonstrated in vitro and in vivo (Culmsee et al., 1999; Zhang et al., 1993).

To examine whether test compounds can promote neuronal survival, hippocampal neurons can be prepared, and the primary neurons pre-treated with various test compounds for a period of time, for example, 30 min, followed by 50 μM glutamate treatment for a period of time, for example, 16 h. A quantitative apoptosis assay with MR(DEVD)2 can be used to determine whether the compounds display a comparable protective effect as the positive control NGF (i.e., apoptosis inhibitory activity).

NGF overexpression decreases infarct volume and neuronal apoptosis in transgenic mice or intraventricular injected mice (Guegan et al., 1998; Luk et al., 2004). NGF also potently protects PC12 cells from apoptosis in an OGD (Oxygen-glucose-deprivation) model (Tabakman et al., 2005). To explore whether test compounds might exert any protective effect on hippocampal neurons in OGD, the primary preparations can be pre-treated with NGF or test compounds 30 min before OGD stimulation. In 3 h, apoptotic analysis can be used to show whether the compounds exhibit potent protective effects. Titration assays can show whether the compounds protect neurons in a dose-dependent manner. Therefore, the assay can identify compounds which selectively protect TrkA expression cells and primary neurons from apoptosis.

Determination of Neurite Outgrowth in PC12 Cells

One of most prominent neurotrophic effects of NGF is to trigger neurite outgrowth in neuronal cells and incur differentiation. To explore whether test compounds possess this activity, PC12 cells can be incubated with a certain concentration of test compounds (for example, 0.5 μM of the compounds) for a period of time, such as 5 days. The cell medium containing the compounds can be replenished periodically, such as every other day.

In this type of assay, NGF will elicit pronounced neurite sprouting in PC12 cells after 5 days of treatment. Those test compounds which are active in one or more of the assays described above, and which also elicit neurite outgrowth in PC12 cells, can be identified as neurotrophic/neuroprotective.

The neurite network generated by a test compound can be used as evidence that the compounds have strong neurotrophic activity. Dose-dependent assays can be used to reveal ideal concentrations to provoke substantial neurite sprouting in PC12 cells, and thus identify compounds which possess potent neurotrophic activity at a concentration comparable to NGF, and robustly provoke neurite outgrowth.

Identification of Compounds Which Trigger TrkA Tyrosine Phosphorylation in Hippocampal Neurons

NGF binds to receptor TrkA and elicits its dimerization and autophosphorylation on tyrosine residues. Numerous tyrosine residues on TrkA are phosphorylated upon NGF stimulation. For example, Y490 phosphorylation is required for Shc association and activation of MAP kinase signaling cascade. Y751 phosphorylation is essential for PI 3-kinase docking and activation.

To evaluate whether test compounds can also trigger TrkA tyrosine phosphorylation, primary hippocampal neurons can be treated with putative compounds at a certain concentration (for example, 0.5 μM), for a certain period of time (for example, 30 min). The cell lysates can be analyzed, for example, by immunoblotting with anti-phospho-TrkA Y490 antibody.

NGF treatment will be shown to induce potent TrkA phosphorylation, and the assay can identify compounds which, like NGF, similarly induce TrkA phosphorylation. TrkA tyrosine phosphorylation in hippocampal neurons can also be demonstrated by immunofluorescent staining with an anti-TrkA Y490 specific antibody.

To determine whether test compounds can trigger TrkA dimerization, GFP-TrkA and HA-TrkA can be cotransfected into HEK293 cells, and the cells treated with 0.5 μM gambogic amide for 30 min. Coimmunoprecipitation assays can be used to determine whether any test compounds provoke TrkA dimerization, ideally even more strongly than NGF, more ideally with a negative control such as DMSO to generate a baseline.

Cotransfected TrkA and TrkB or TrkC receptors will fail to dimerize regardless of pre-treatment with NGF or with compounds that also selectively trigger dimerization of the TrkA receptors. Selective TrkA agonists will elicit tyrosine phosphorylation in TrkA, but not in TrkB or C receptors (or in cells co-transfected to express two or more of these receptors, i.e., TrkA in combination with TrkB and/or TrkC).

Some compounds may be effected in such a manner that TrkA-KD will display decreased tyrosine phosphorylation compared to wild-type TrkA, which will indicate that not only TrkA autophosphorylation but also other tyrosine kinases are activated, and contribute to Y490 phosphorylation. These assays can thus identify compounds which mimic NGF and selectively provoke TrkA dimerization and tyrosine phosphorylation.

Identification of Compounds Which Provokes Akt and MAP Kinase Activation

NGF triggers PI 3-kinase/Akt and Ras/MAP Kinase signaling cascades activation through the TrkA receptor. To explore whether test compounds possess similar mitogenic effects, T17 cells can be treated with various test compounds for 30 min. The cell lysates can be analyzed by immunoblotting with anti-phospho-Erk1/2 and phospho-Akt-473 antibodies, respectively.

NGF treatment stimulates demonstrable Erk1/2 and Akt phosphorylation. Those compounds which similarly provoke robust phosphorylation of both Erk1/2 and Akt will be shown to be effective TrkA agonists (or partial agonists).

Compounds which are unable to activate MAP kinase, but which strongly provoke Akt activation, might differentially regulate PI 3-kinase/Akt and Ras/MAP kinase pathways either through the TrkA receptor or its downstream cellular targets.

The assays can be extended into primary hippocampal neurons. NGF and certain test compounds may activate Akt, while other compounds may slightly upregulate Akt phosphorylation. Time course assays with hippocampal neurons can be used to show that certain compounds elicit Akt phosphorylation after 5 min treatment, and whether Akt activation increases over time, whether it can be sustained, and whether it provokes Akt activation in a dose-dependent manner. Taken together, this information can identify compounds which mimic NGF and potently activate Akt and MAP kinase activation in neurons.

Identification of Compounds Which Bind the Cytoplasmic Juxtamembrane Domain of TrkA Receptor

The immunoglobulin (Ig)-like domain (TrkA-d5 domain) in the extracellular region of TrkA proximal to the membrane is required for specific binding of NGF (Urfer et al., 1995). To investigate which portion of TrkA receptor binds to test compounds that also appear to be TrkA agonists, based on the assays described above, in vitro binding assay can be conducted with immobilized compounds by covalently linking the compounds to a solid substrate, such as affi-gel 102.

GFP-tagged TrkA truncates can be prepared, and transfected into HEK293 cells. Binding assays can be used to show whether the extracellular domain is or is not required for the association. The cytoplasmic juxtamembrane domain is critical for ligand binding. Although the intracellular domain (ICD) of Trk family members shares great homology, the juxtamembrane region varies. In vitro binding assay can be used to show whether test compounds selectively bound to wild-type and/or kinase-dead TrkA, but not to TrkB or TrkC. Thus, selective TrkA agonists (or partial agonists) can be identified.

If test compounds have a weaker affinity to Trk-KD than to wild-type TrkA, and/or fail to bind to p75NTR, ErbB3 or EGF receptors, this can show that the test compounds specifically associate with TrkA but not other neurotrophin receptors or transmembrane tyrosine kinase receptors.

To determine the binding constant between test compounds and TrkA, one can conduct a competition assay with GFP-TrkA-bound beads covalently linked to the test compounds. If the concentration of bead-associated TrkA is gradually decreased as the free concentration of test compounds increases, quantitative analysis of the competition data will show the K_{d} of the test compounds. Incubation of FITC-conjugated test compounds with PC12 cells for 10 minutes can elicit their association with TrkA receptors, whereas FITC alone fails to penetrate into cells. Using this information, one can determine whether a test compound penetrates the cell membrane and binds tightly to TrkA receptor through the same or a different region than NGF.

Determination of Whether a Test Compound Can Prevent Kainic Acid-Triggered Neuronal Apoptosis and Decrease Infarct Volume in Stroked Rat Brain

Kainic acid (KA) is a potent agonist for the AMPA receptor. Peripheral injections of KA result in recurrent seizures and the subsequent degeneration of select populations of neurons in the hippocampus (Nadler et al., 1980; Schauwecker and Steward, 1997; Sperk et al., 1983). It has been shown that the activation of caspase-3 is a necessary component of KA-induced cell death (Faherty et al., 1999). To explore whether test compounds can block the neurotoxicity initiated by KA, the compounds can be subcutaneously injected (for example, at a concentration of 2 mg/kg) into C57BL/6 mice, followed by 25 mg/kg KA. In 5 days, the mice can be perfused and the brains cut to a thickness of 5 μm and mounted on slides. In the absence of a neuroprotective compound, TUNEL staining reveals that KA provokes enormous apoptosis in the hippocampus. If this apoptosis is substantially diminished by a test compound, this assay can demonstrate that the test compound is neuroprotective.

To further determine the neuroprotective potential in vivo, test compounds can be tested in a transient middle cerebral artery occlusion (MCAO) stroke model in adult male rats. After 2 h MCAO followed by reperfusion, the animals receive vehicle or test compounds (2 mg/kg) 5 min prior to the onset of reperfusion. If all or a substantial number of test animals survive the ischemic insult and treatment with the test compound, this will demonstrate that the compound is neuroprotective.

A representative brain slice stained with TTC 24 h after MCAO in vehicle-treated and compound-treated rats can be subjected to area and volume measurements from TTC sections to indicate whether treatments with the compound substantially reduces infarct volumes in this transient ischemic model of stroke. Positive results can be compared with those of NGF, which is known to reduce infarct volume and apoptosis in focal ischemia (Guegan et al., 1998).

LDF (laser-Doppler flowmetry) can be measured over the ipsilateral parietal cortex. One can measure the reduction of relative CBF (cerebral blood flow) within 5 minutes of MCAO in rats that subsequently received test or vehicle treatment.

Taken together, the assays described above can identify potent TrkA agonists (or partial agonists), which are capable of preventing neuronal cell death and protecting the neurodegeneration elicited by excitatory neurotoxicity and stroke.

### II. Methods of Providing Neuroprotection Using TrkA Agonists

The selective TrkA agonists can be used to treat or prevent neurodegenerative disorders. The method involves administering to an animal an effective amount of a selective TrkA agonist, or a pharmaceutically acceptable salt or prodrug of the selective TrkA agonist, to provide neuroprotection, and to treat or prevent neurodegenerative diseases.

Selective TrkA agonists and partial agonists, such as those identified using the screening process described above, can be used to provide neuroprotection, before, during, or after a neural insult, such as a stroke or other ischemic event. The compounds neuroprotective effects can prevent loss of neuronal cell viability induced by exitotoxic agents in regions involved in memory encoding and exhibiting early degeneration in Alzheimer's disease and ischemia.

The selective TrkA agonists and partial agonists can also be used to treat demyelination disorders such as MS. While not wishing to be bound to a particular theory, it is believed that the selective TrkA agonists treat or prevent autoimmune demyelination, such as that observed in multiple sclerosis (MS), by inducing myelin protein genes.

The selective TrkA agonists and partial agonists can further be used to treat or prevent neurodegenerative disorders such as epilepsy, head and spinal chord trauma, Parkinson disease, Huntington's disease, Alzheimer's disease, or amyotrophic lateral schlerosis, or a neurological disorder. Additional neurological diseases that can be treated with the selective TrkA agonists are movement disorders, pain and the like.

The selective TrkA agonists or partial agonists can further be used to promote nerve cell survival, and can protect neural cells against cell death, for example, cell death due to the effects of neurotoxic agents.

In each of these methods, the TrkA agonist or partial agonists, or pharmaceutically acceptable salt or prodrug thereof, are administered to an animal an effective amount, optionally in a pharmaceutical composition, to provide neuroprotection, to treat demyelinating diseases, to enhance nerve regeneration, and/or to treat or prevent neurodegenerative diseases.

The compounds offer advantages over NGF, in that they are small molecules, and can be administered orally and prepared relatively inexpensively and in relatively high purity, relative to NGF. Further, the compounds are more selective than NGF, by binding selectively to TrkA, rather than non-selectively to TrkA, TrkB, and TrkC.

### IV. Pharmaceutical Compositions

The compounds described herein can be incorporated into pharmaceutical compositions and used to prevent a condition or disorder in a subject susceptible to such a condition or disorder, and/or to treat a subject suffering from the condition or disorder. The pharmaceutical compositions described herein include one or more of the honokiol analogues described herein, and/or pharmaceutically acceptable salts thereof. Optically active compounds can be employed as racemic mixtures, as pure enantiomers, or as compounds of varying enantiomeric purity.

The manner in which the compounds are administered can vary. The compositions are preferably administered orally (e.g., in liquid form within a solvent such as an aqueous or non-aqueous liquid, or within a solid carrier). Preferred compositions for oral administration include pills, tablets, capsules, caplets, syrups, and solutions, including hard gelatin capsules and time-release capsules. Compositions may be formulated in unit dose form, or in multiple or subunit doses. Preferred compositions are in liquid or semisolid form. Compositions including a liquid pharmaceutically inert carrier such as water or other pharmaceutically compatible liquids or semisolids may be used. The use of such liquids and semisolids is well known to those of skill in the art.

The compositions can also be administered via injection, i.e., intraveneously, intramuscularly, subcutaneously, intraperitoneally, intraarterially, intrathecally; and intracerebroventricularly. Intravenous administration is a preferred method of injection. Suitable carriers for injection are well known to those of skill in the art, and include 5% dextrose solutions, saline, and phosphate buffered saline. The compounds can also be administered as an infusion or injection (e.g., as a suspension or as an emulsion in a pharmaceutically acceptable liquid or mixture of liquids).

The formulations may also be administered using other means, for example, rectal administration. Formulations useful for rectal administration, such as suppositories, are well known to those of skill in the art. The compounds can also be administered by inhalation (e.g., in the form of an aerosol either nasally or using delivery articles of the type set forth in U.S. Pat. No. 4,922,901 to Brooks et al., the disclosure of which is incorporated herein in its entirety); topically (e.g., in lotion form); or transdermally (e.g., using a transdermal patch, using technology that is commercially available from Novartis and Alza Corporation). Although it is possible to administer the compounds in the form of a bulk active chemical, it is preferred to present each compound in the form of a pharmaceutical composition or formulation for efficient and effective administration.

The compounds can be incorporated into drug delivery devices such as nanoparticles, microparticles, microcapsules, and the like. Representative microparticles/nanoparticles include those prepared with cyclodextrins, such as pegylated cyclodextrins, liposomes, including small unilamellar vesicles, and liposomes of a size designed to lodge in capillary beds around growing tumors. Suitable drug delivery devices are described, for example, in Heidel J D, et al., Administration in non-human primates of escalating intravenous doses of targeted nanoparticles containing ribonucleotide reductase subunit M2 siRNA, Proc Natl Acad Sci USA. 2007 Apr. 3; 104(14):5715-21; Wongmekiat et al., Preparation of drug nanoparticles by co-grinding with cyclodextrin: formation mechanism and factors affecting nanoparticle formation, Chem Pharm Bull (Tokyo). 2007 March; 55(3):359-63; Bartlett and Davis, Physicochemical and biological characterization of targeted, nucleic acid-containing nanoparticles, Bioconjug Chem. 2007 March-April; 18(2): 456-68; Villalonga et al., Amperometric biosensor for xanthine with supramolecular architecture, Chem Commun (Camb). 2007 Mar. 7; (9):942-4; Defaye et al., Pharmaceutical use of cyclodextrines: perspectives for drug targeting and control of membrane interactions, Ann Pharm Fr. 2007 January; 65(1):33-49; Wang et al., Synthesis of Oligo(ethylenediamino)-beta-Cyclodextrin Modified Gold Nanoparticle as a DNA Concentrator; Mol Pharm. 2007 March-April; 4(2):189-98; Xia et al., Controlled synthesis of Y-junction polyaniline nanorods and nanotubes using in situ self-assembly of magnetic nanoparticles, J Nanosci Nanotechnol., 2006 December; 6(12):3950-4; and Nijhuis et al., Room-temperature single-electron tunneling in dendrimer-stabilized gold nanoparticles anchored at a molecular printboard, Small. 2006 December; 2(12):1422-6.

Exemplary methods for administering such compounds will be apparent to the skilled artisan. The usefulness of these formulations may depend on the particular composition used and the particular subject receiving the treatment. These formulations may contain a liquid carrier that may be oily, aqueous, emulsified or contain certain solvents suitable to the mode of administration.

The compositions can be administered intermittently or at a gradual, continuous, constant or controlled rate to a warm-blooded animal (e.g., a mammal such as a mouse, rat, cat, rabbit, dog, pig, cow, or monkey), but advantageously are administered to a human being. In addition, the time of day and the number of times per day that the pharmaceutical formulation is administered can vary.

Preferably, the compositions are administered such that active ingredients interact with regions where cancer cells are located. The compounds described herein are very potent at treating these cancers.

In certain circumstances, the compounds described herein can be employed as part of a pharmaceutical composition with other compounds intended to prevent or treat a particular cancer, i.e., combination therapy. In addition to effective amounts of the compounds described herein, the pharmaceutical compositions can also include various other components as additives or adjuncts.

Combination Therapy

The combination therapy may be administered as (a) a single pharmaceutical composition which comprises a selective TrkA agonist or partial agonist as described herein, for example, gambogic amine, at least one additional pharmaceutical agent described herein, and a pharmaceutically acceptable excipient, diluent, or carrier; or (b) two separate pharmaceutical compositions comprising (i) a first composition comprising a selective TrkA agonist or partial agonist as described herein and a pharmaceutically acceptable excipient, diluent, or carrier, and (ii) a second composition comprising at least one additional pharmaceutical agent described herein and a pharmaceutically acceptable excipient, diluent, or carrier. The pharmaceutical compositions can be administered simultaneously or sequentially and in any order.

When used to treat demyelination disorders such as MS, the selective TrkA agonist or partial agonist can be administered with other compounds known to treat MS, such as interferon and pegylated interferon (Pegasys).

When used to treat or prevent neurodegenerative disorders such as AD and Parkinson's disease, the selective TrkA agonist or partial agonist can be administered with other compounds known to treat such disorders, such as dopamine and Aricept®.

When used to provide neuroprotection, the selective TrkA agonist or partial agonist can be administered with other compounds known to provide neuroprotection, such as adenosine (Dall'lgna et al., “Neuroprotection by caffeine and adenosine A_{2A} receptor blockade of -amyloid neurotoxicity,” British Journal of Pharmacology (2003) 138, 1207-1209).

The following examples are illustrative, but not limiting, of the method and compositions of the present invention. Other suitable modifications and adaptations of the variety of conditions and parameters normally encountered in clinical therapy, and which are obvious to those skilled in the art, are within the spirit and scope of the invention.

### EXAMPLE 1

### Gambogic Acid

Procedure 1:

Step A.

The dry gamboge powder (140 g) was extracted with MeOH (3.times.600 mL) at room temperature for 1 week, after filtration, the solvent was removed under reduced pressure, gave crude extract (122 g) as yellow powder.

Step B. Gambogic Acid Pyridine Salt.

The above crude extract (120 g) was dissolved in pyridine (120 mL), then warm water (30 mL) was added to the stirred solution. After cooling to r.t., some precipitate was observed. Hexane (120 mL) was added to the mixture and the mixture was filtered and the solid was washed with hexane and dried. The salt was purified by repeated recrystallization from ethanol and gave gambogic acid pyridine salt (7₅ g); HPLC: 99%.

Step C. Gambogic Acid.

The gambogic acid pyridine salt (0.4 g) was dissolved in ether (25 mL) and shaken with aqeuous HCl (1N, 25 mL) for 1 h. The ether solution was then washed with water (2.times₁0 mL), dried and evaporated to give the title compound (345 mg); HPLC: 99%. ¹H NMR (CDCl₃): 12.66 (s, 1H), 7.43 (d, J=6.9 Hz, 1H), 6.48 (d, J=10.2 Hz, 1H), 5.97 (t, J=7₅ Hz, 1H), 5.26 (d, J=9.9 Hz, 1H), 4.91 (m, 2H), 3.37 (m, 1H), 3.24-2.98 (m, 2H), 2.81 (d, J=6.6 Hz, 1H), 2.41 (d, J=9 Hz, 1H), 2.20 (m, J₁=8.4 Hz, J₂=5₁ HZ, 1H), 1.88 (m, 1H), 1.63 (s, 3H), 1.60 (s, 3H), 1₅8 (s, 3H), 1₅3 (s, 3H), 1₅1 (s, 3H), 1.43 (s, 3H), 1.26 (s, 3H), 1₁8 (s, 3H). MS: 627 (M−H).

Procedure 2:

The crude extract of gamboge (300 mg) was purified by repeated column chromatography (SiO₂, hexane-EtOAc gradient) using a Combi Flash SG 100 separation system, gave 18 mg of gambogic acid; HPLC: 94%, MS. 627 (M−H).

The gambogic acid thus prepared can be used to prepare gambogic amines, for example, using the chemistry outlined above in Scheme 1.

### EXAMPLE 2

### A Cell-Based Screen for Protecting TrkA Expressing Cells From Apoptosis

In order to identify small molecules that mimic NGF and activate TrkA, we developed a cell-based apoptotic assay using a cell permeable fluorescent dye MR(DERD)2, which turns red upon caspase-3 cleavage in apoptotic cells. We utilized a murine cell line T17, which was derived from basal forebrain SN56 cells. T17 cells are TrkA stably transfected SN56 cells. The candidates selectively protecting T17 but not SN56 cells from the first round screen can then be subjected to neurite outgrowth assay for the secondary screen. The positive compounds can be analyzed for TrkA tyrosine phosphorylation, Akt and MAP kinases signaling cascade activation.

T17 cells can be cultured in 96-well plates and preincubated with 10 μM compounds for 30 min, followed by 1 μM staurosporine (STS) treatment for 9 h. MR(DEVD)2 can be introduced to the cells 1 h before examination under fluorescent microscope. The apoptotic cells are red, while live cells have no signal. As a positive control, NGF substantially decreases the red cell numbers compared to DMSO control. Using the caspase-3-activated fluorescent dye as a visual assay, one can screen the gambogic amine compounds.

Those compounds which selectively protect T17 but not SN56 cells from STS-initiated apoptosis might act either directly through TrkA receptor or its downstream signaling effectors. Even in the absence of NGF, T17 exhibits a stronger anti-apoptotic effect than its parental SN56 cells, indicating that overexpression of TrkA suppresses caspase-3 activation. NGF treatment further enhances this effect.

Identification of Gambogic Amines as Survival Enhancers

To compare the apoptosis inhibitory activity of various compounds, one can pre-incubate gambogic amines derivatives (0.5 μM) with T17 and SN56 cells, followed by 1 μM STS for 9 h. Quantitative analysis of the apoptosis inhibitory activities can determine whether the compounds protect SN56 cells from apoptosis. As controls, gambogic acid, gambogic amide, dimethyl gambogate, and dihydrogambogic acid strongly suppress apoptosis in T17 cells with protective activities even stronger than NGF. These findings suggest that some gambogic acid derivatives can trigger programmed cell death, in agreement with a previous report that they possess anti-cancer activity (Kasibhatla et al., 2005).

TrkA is highly expressed in hippocampal neurons (Culmsee et al., 2002; Kume et al., 2000; Zhang et al., 1993). TrkA and p75NTR are up-regulated in hippocampal and cortical neurons under pathophysiological conditions (Kokaia et al., 1998; Lee et al., 1998). Moreover, neuroprotective effects of NGF in hippocampal and cortical neurons have been demonstrated in vitro and in vivo (Culmsee et al., 1999; Zhang et al., 1993). To examine whether these compounds can promote neuronal survival, one can prepare hippocampal neurons and pre-treat the primary neurons with various gambogic amine derivatives for 30 min, followed by 50 μM glutamate treatment for 16 h. Quantitative apoptosis assay with MR(DEVD)2 can demonstrate whether the gambogic amines displaye the same protective effect as the positive control NGF.

NGF overexpression decreases infarct volume and neuronal apoptosis in transgenic mice or intraventricular injected mice (Guegan et al., 1998; Luk et al., 2004). NGF also potently protects PC12 cells from apoptosis in an OGD (Oxygen-glucose-deprivation) model (Tabakman et al., 2005). To explore whether gambogic amines exert any protective effect on hippocampal neurons in OGD, one can pre-treat the primary preparations with NGF or various compounds 30 min before OGD stimulation. In 3 h, apoptotic analysis can show whether the gambogic amines exhibit potent protective effects. Titration assays can reveal whether or not the compounds protect neurons in a dose-dependent manner. The data can identify those gambogic amines which selectively protect TrkA expression cells and primary neurons from apoptosis.

Elicitation of Neurite Outgrowth in PC12 Cells

One of most prominent neurotrophic effects of NGF is to trigger neurite outgrowth in neuronal cells and incur differentiation. To explore whether gambogic amine compounds possess this activity, one can incubate PC12 cells with 0.5 μM compounds for 5 days. The cell medium containing the compounds can be replenished every other day. NGF elicits pronounced neurite sprouting in PC12 cells after 5 days of treatment. The gambogic amine compounds can similarly be evaluated for their ability to induce evident neurite outgrowth in PC12 cells. Dose-dependent assays can reveal whether 10-50 nM of gambogic amines are sufficient to provoke substantial neurite sprouting in PC12 cells. Thus, one can determine whether the gambogic amines possess potent neurotrophic activity at a concentration comparable to NGF, and robustly provoke neurite outgrowth.

Triggering of TrkA Tyrosine Phosphorylation in Hippocampal Neurons

NGF binds to receptor TrkA and elicits its dimerization and autophosphorylation on tyrosine residues. Numerous tyrosine residues on TrkA are phosphorylated upon NGF stimulation. For example, Y490 phosphorylation is required for Shc association and activation of MAP kinase signaling cascade. Y751 phosphorylation is essential for PI 3-kinase docking and activation. To evaluate whether gambogic amine compounds can also trigger TrkA tyrosine phosphorylation, one can treat primary hippocampal neurons with various drugs at 0.5 μM for 30 min. The cell lysates can be analyzed by immunoblotting with anti-phospho-TrkA Y490 antibody. NGF treatment induces potent TrkA phosphorylation, and the gambogic amines can be evaluated for their ability to similarly induce this effect. One can also determine whether gambogic amines initiate pronounced TrkA tyrosine phosphorylation in hippocampal neurons, as demonstrated by immunofluorescent staining with anti-TrkA Y490 specific antibody. To explore whether gambogic amines can trigger TrkA dimerization, one can cotransfect GFP-TrkA and HA-TrkA into HEK293 cells, and treat the cells with 0.5 μM gambogic amine for 30 min. Coimmunoprecipitation assays can be used to determine whether gambogic amines provoke TrkA dimerization even more strongly than NGF. One can also evaluate whether the gambogic amines also elicit tyrosine phosphorylation in TrkA but not in TrkB or C receptors. Hence, one can determine whether the gambogic amines mimic NGF and selectively provoke TrkA dimerization and tyrosine phosphorylation.

Provoking Akt and MAP kinase Activation

NGF triggers PI 3-kinase/Akt and Ras/MAP Kinase signaling cascades activation through the TrkA receptor. To explore whether the gambogic amine derivatives possess similar mitogenic effects, one can treat T17 cells with various gambogic acid derivatives for 30 min. The cell lysates can be analyzed by immunoblotting with anti-phospho-Erk1/2 and phospho-Akt-473 antibodies, respectively. NGF treatment stimulates demonstrable Erk1/2 and Akt phosphorylation, and the gambogic amines can be evaluated for their ability to do so.

One can also extend the assays into primary hippocampal neurons. NGF evidently activates Akt, and the gambogic amines' ability to do so can be evaluated. Time course assay with hippocampal neurons can show whether gambogic amines (0.5 μM) elicit Akt phosphorylation after 5 min treatment, and whether Akt activation increases after 10 min and is sustained for 60 min. One can also determine whether the gambogic amines provoke Akt activation in a dose-dependent manner. Taken together, these observations can demonstrate that the gambogic amines mimic NGF and potently activate Akt and MAP kinases activation in neurons.

Binding the Cytoplasmic Juxtamembrane Domain of TrkA Receptor

The immunoglobulin (Ig)-like domain (TrkA-d5 domain) in the extracellular region of TrkA proximal to the membrane is required for specific binding of NGF (Urfer et al., 1995). To investigate which portion of TrkA receptor binds to gambogic amines, one can conduct in vitro binding assay with immobilized gambogic amines through covalent linkage of affi-gel 102. For use in the assay, we generated numerous GFP-tagged TrkA truncates and transfected them into HEK293 cells. Binding assays showed that the extracellular domain was not required for the association. Interestingly, the cytoplasmic juxtamembrane domain was critical for the ligand binding. Although the intracellular domain (ICD) of Trk family members shares great homology, the juxtamembrane region varies. In vitro binding assays can reveal whether the gambogic amines selectively bind to both wild-type and kinase-dead TrkA but not to TrkB or TrkC. If Trk-KD exhibits weaker affinity to the gambogic amine than wild-type TrkA, and the amine does not bind to p75NTR, ErbB3 or EGF receptors, such information would underscore the finding that gambogic amines specifically associate with TrkA but not other neurotrophin receptors or transmembrane tyrosine kinase receptors. To determine the binding constant between a gambogic amine and TrkA, one can conduct a competition assay with GFP-TrkA-bound gambogic amine beads. Quantitative analysis of the competition data can be used to reveale the K_{d}. Incubation of FITC-conjugated gambogic amine with PC12 cells for 10 min can be used to elicit information on its association with the TrkA receptor, whereas FITC alone fails to penetrate into cells. Thus, the information can show whether a gambogic amine penetrates the cell membrane and binds tightly to TrkA receptor through a different region from NGF.

Prevention of Kainic Acid-Triggered Neuronal Apoptosis and Decreased Infarct Volume in Stroked Rat Brain

Kainic acid (KA) is a potent agonist for the AMPA receptor. Peripheral injections of KA result in recurrent seizures and the subsequent degeneration of select populations of neurons in the hippocampus (Nadler et al., 1980; Schauwecker and Steward, 1997; Sperk et al., 1983). It has been shown that the activation of caspase-3 is a necessary component of KA-induced cell death (Faherty et al., 1999). To explore whether gambogic amines can block the neurotoxicity initiated by KA, one can subcutaneously inject 2 mg/kg of a gambogic amine into C57BL/6 mice, followed by 25 mg/kg KA. In 5 days, the mice can be perfused and the brains were cut to a thickness of 5 μm and mounted on slides. TUNEL staining can reveal whether KA provokes enormous apoptosis in the hippocampus, and whether this effect is substantially diminished by the gambogic amine. Quantitative analysis of apoptosis in the hippocampus revealed that KA induced 47% and 57% cell death in the CA1 and CA3 region, and the results can be compared with those of the gambogic amine.

To further determine the neuroprotective potential in vivo, the gambogic amine can be tested in a transient middle cerebral artery occlusion (MCAO) stroke model in adult male rats. After 2 h MCAO followed by reperfusion, the animals can receive vehicle or the gambogic amine (2 mg/kg) 5 min prior to the onset of reperfusion. If all or a significant number of the animals included in the study survive the ischemic insult and treatment with the gambogic amine, it will demonstrate the neuroprotective potential of the gambogic amine. Area and volume measurements from TTC sections can indicate whether treatments with the gambogic amine substantially reduces infarct volumes in this transient ischemic model of stroke. If so, these results can be compared with a previous report that NGF reduces infarct volume and apoptosis in focal ischemia (Guegan et al., 1998). LDF (laser-Doppler flowmetry) can be measured over the ipsilateral parietal cortex. The effects of the gambogic amine on relative CBF (cerebral blood flow) can be evaluated. After filament withdrawal (120 min), relative CBF can also be measured in the vehicle-treated group and gambogic amine-treated groups, and compared with preischemic levels. For those gambogic amines in which there are no significant differences between the groups, the data will suggest that the relative ischemic insult was equivalent among all groups.

Mounting evidence demonstrates that Trk family members play a crucial role in initiation, progression, and metastasis of many tumors in humans (Descamps et al., 2001; Douma et al., 2004; McGregor et al., 1999). Members of the neurotrophin receptor family are up-regulated in a variety of human cancers, including prostate (Weeraratna et al., 2000), pancreatic (Zhang et al., 2005) and breast cancers. For instance, NGF strongly stimulates breast cancer cell growth, which is mediated by TrkA and p75NTR respectively (Dolle et al., 2004). Emerging evidence demonstrates that TrkA plays a key role in the progression of these cancers. In the case of pancreatic cancer, increased expression of TrkA also correlates with an increased level of pain. Staurosporine Trk inhibitors from Cephalon Pharmaceuticals have shown excellent preclinical anti-tumor efficacy (George et al., 1999) and have entered human clinical trials (Lippa et al., 2006; Marshall et al., 2005). Since TrkA abnormal activation in numerous tumors contributes to cancer progression, small molecules like gambogic amines might not only prevent neuronal cells from death, they might also promote cancer proliferation as well.

Gambogic acid has been used in traditional Chinese medicine to treat cancers. It potently blocks human cancer proliferation in vitro and in animals (Wu et al., 2004; Zhang et al., 2004; Zhao et al., 2004). Recently, it has been shown that the Transferrin receptor functions as a cellular target for gambogic acid to exert its anticancer activity. Presumably, gambogic amines might bind Transferrin receptor as well as TrkA. Gambogic acid associates with Transferrin with K_{d} of 2.2 μM (Kasibhatla et al., 2005). Conceivably, gambogic amines will bind to TrkA receptor much more specifically and tightly than to Transferrin receptor. Consequently, they might exert neurotrophic activity more robustly and selectively than apoptotic effects. NGF regulates neuronal apoptosis through the action of critical protein kinase cascades, such as the phosphoinositide 3-kinase/Akt and mitogen-activated protein kinase pathways. Since neurodegeneration is an underlying cause of various nervous system disorders, including Alzheimer's disease and amyotrophic lateral sclerosis, it is important that molecules, which provide trophic support for neurons, are identified, and their mechanisms of action defined. In addition to its role as target-derived survival factors, NGF also modulates activity-dependent neuronal plasticity in adult neurons. Moreover, NGF may be useful for the treatment of neurodegenerative disorders such as Alzheimer's disease (Olson, 1993). Thus, those gambogic amines which are TrkA agonists (and partial agonists) and prevent neuronal cell death may be clinically important for the treatment of various neurodegenerative diseases and stroke.

Materials and Methods

Cells and Reagents

PC12 cells were maintained in medium A (DMEM with 10% fetal bovine serum (FBS), 5% horse serum and 100 units penicillin-streptomycin) at 37° C. with 5% CO₂ atmosphere in a humidified incubator. Mouse septal neuron×neuroblastoma hybrids SN56 cells were created by fusing N18TG2 neuroblastoma cells with murine (strain C57BL/6) neurons from postnatal 21 days septa. SN56 cells were maintained at 37° C. with 5% CO₂ atmosphere in DMEM medium containing 1 mM pyruvate and 10% FBS. T17 cells, stably transfected with rat TrkA were cultured in the same medium containing 300 μg/ml G418. The cells are gifts from Dr. Brygida Berse at Boston University. NGF was from Roche. Phospho-Akt-473 or 308, Akt and lamin A/C antibodies were from Cell Signaling. Anti-phospho-Erk1/2, anti-phospho-TrkA Y490, and anti-phospho-Akt 473 antibodies were from Upstate Biotechnology, Inc. The chemical library containing 2000 biologically active compounds was from The Spectrum Collection (MicroSource Discovery System, Inc. Gaylordsville, Conn. 06755). All chemicals not included above were purchased from Sigma.

Cell-Based Screen

T17 cells can be seeded in a 96-well plate at 10,000 cells/well in 100 μl complete medium. Cells can be incubated overnight, followed by 30 min pretreatment with 10 μM compounds in DMSO (10 mM stock concentration from The Spectrum Collection library). The cells can then be treated with 1 μM Staurosporine for 9 h. One h before the termination of the experiment, 10 μM MR(DEVD)2, a cell permeable caspase-3-activated fluorescent dye can be introduced. Cells can be fixed with 4% paraformaldehyde for 15 min. Cells can then be washed with PBS and incubated with 1 μg/ml of Hoechst 33342 for 10 min. Cover slides can then be washed with PBS, mounted, and examined using a fluorescence microscope.

Immobilization of Gambogic Amines, and Synthesis of FITC-Gambogic Amines

Gambogic amines can be immobilized to a suitable affinity gel (such as Profinity™ Epoxide Resin by Bio-Rad) which contains epoxide groups reactive with the amine group on the gambogic amines. The reaction can be carried out at room temperature. The reaction mixture can be washed to remove excess reagents, and the gambogic amine-conjugated beads can be kept in 1×PBS at 4° C. in the dark. The isocyanate form of fluorescein, FITC, can be conjugated with gambogic amines The amines can be introduced into FITC in a suitable solvent, such as ethanol. After 2 h incubation at room temperature, the reaction solution can be poured into water, then extracted with ethyl acetate. The organic layers can be dried and concentrated to yield the crude product, which can be purified by chromatography (SiO₂, EtOAc-hexane) to yield the desired compounds.

Co-Immunoprecipitation and In Vitro Binding Assays

A 10-cm plate of HEK293 cells or PC12 cells can be washed once in PBS, and lysed in 1 ml lysis buffer (50 mM Tris, pH 7.4, 150 mM NaCl, 1 mM EDTA, 0.5% Triton X-100, 1.5 mM Na₃VO₄, 50 mM NaF, 10 mM sodium pyrophosphate, 10 mM sodium β-glycerophosphate, 1 mM phenylmethylsulfonyl flouride (PMSF), 5 mg/ml aprotinin, 1 mg/ml leupeptin, 1 mg/ml pepstatin A), and centrifuged for 10 min at 14,000×g at 4° C. The supernatant can be transferred to a fresh tube. After SDS-PAGE, the samples were transferred to a nitrocellular membrane. Western blotting analysis can be performed with a variety of antibodies.

Kainic Acid/Gambogic Amine Drug Administration

Male C57BL/6 mice aged of 60 days can be injected subcutaneously (s.c.) with a single dose of either 30% ethanol in saline or KA (25 mg/kg) (Sigma, Mo.) or the gambogic amine (2 mg/kg) followed by KA. Animals can be continually monitored for 2 h for the onset of seizure activity. At 5 days following treatment, animals can be anesthetized and perfused with 4% paraformaldehyde in 0.1 M phosphate buffered saline. Brains can be removed, post-fixed overnight and processed for paraffin embedding. Serial sections can be cut at 5 μm and mounted on slides (Superfrost-plus, Fisher). The slides can be processed for TUNEL staining in order to assess the degree of DNA fragmentation.

Focal Ischemia Model

Rats can be used in a study of focal ischemia. Criteria for inclusion/exclusion of rats from the study group can be based on the laser-Doppler flowmetry (LDF) measurement of cerebral blood flow (CBF). To ensure relative uniformity of the ischemic insult, animals with mean ischemic LDF >40% of baseline LDF can be excluded from the cohort. This procedure results in consistently larger and more uniform infarcts, reducing experimental variablity. Anesthesia can be induced by inhalation of 5% isoflurane (in a N₂/O₂ 70%/30% mixture) and maintained by inhalation of 2% isoflurane. Using a SurgiVet (model V3304; Waukesha, Wis., USA) pulse oximeter, blood SpO₂ can be monitored and maintained at levels ≧90%. Body temperature can be monitored throughout surgery (via rectal probe) and maintained at 36.5° C. to 37.5° C. using a heating blanket (Harvard Apparatus, South Natick, Mass., USA). A small incision can be made in the skin overlying the temporalis muscle and the laser-Doppler probe (Moor Instruments, Wilmington, Del., USA) can be positioned on the superior portion of the temporal bone (6 mm lateral and 2 mm posterior from bregma). Focal cerebral ischemia can be induced by occlusion of the right middle cerebral artery as previously described (Sayeed et al., 2006). Lesion Volume: The rats can be sacrificed 24 h post-occlusion with an overdose (75 mg/kg) of Nembutal sodium solution. The brains can be carefully removed and placed in ice-cold saline, and then sliced into 7 serial coronal sections of 2 mm thickness with a rat brain matrix (Harvard Apparatus) starting at 1 mm posterior to the anterior pole. After sectioning, the slices can be stained with 2% 2,3,5-triphenyltetrazolium chloride (TTC; Sigma) in saline and kept for 10 min at 37° C. in the dark. Stained sections can then be fixed in 10% buffered formalin. Both hemispheres of each stained coronal section can be scanned using a high-resolution scanner (Epson Perfection 2400 Photo), and then evaluated by digital image analysis (Image Pro System, Media Cybernetics, Silver Spring, Md., USA). Drug Administration: The rats subjected to MCAO incurring ischemic insult <40% of baseline LDF can be randomly assigned to receive either GA (n=4), or vehicle (n=4) treatment. GA can be given in the amount of 2 mg/kg by ip injection 5 min prior to the onset of reperfusion. Rats in the vehicle group underwent the same experimental protocol, except that they received an identical volume/weight of vehicle only.

### REFERENCES

Arevalo, J. C., Conde, B., Hempstead, B. L., Chao, M. V., Martin-Zanca, D., and Perez, P. (2000). TrkA immunoglobulin-like ligand binding domains inhibit spontaneous activation of the receptor. Mol Cell Biol 20, 5908-5916.

Asano, J., Chiba, K., Tada, M., and Yoshii, T. (1996). Cytotoxic xanthones from Garcinia hanburyi. Phytochemistry 41, 815-820.

Backman, C., Rose, G. M., Bartus, R. T., Hoffer, B. J., Mufson, E. J., and Granholm, A. C. (1997). Carrier mediated delivery of NGF: alterations in basal forebrain neurons in aged rats revealed using antibodies against low and high affinity NGF receptors. J Comp Neurol 387, 1-11.

Barinaga, M. (1994). Possible new test found for Alzheimer's disease. Science 266, 973.

Carter, B. D., and Lewin, G. R. (1997). Neurotrophins live or let die: does p75NTR decide? Neuron 18, 187-190.

Connor, B., and Dragunow, M. (1998). The role of neuronal growth factors in neurodegenerative disorders of the human brain. Brain Res Brain Res Rev 27, 1-39.

Cortazzo, M. H., Kassis, E. S., Sproul, K. A., and Schor, N. F. (1996). Nerve growth factor (NGF)-mediated protection of neural crest cells from antimitotic agent-induced apoptosis: the role of the low-affinity NGF receptor. J Neurosci 16, 3895-3899.

Counts, S. E., Nadeem, M., Wuu, J., Ginsberg, S. D., Saragovi, H. U., and Mufson, E. J. (2004). Reduction of cortical TrkA but not p75(NTR) protein in early-stage Alzheimer's disease. Ann Neurol 56, 520-531.

Culmsee, C., Gerling, N., Lehmann, M., Nikolova-Karakashian, M., Prehn, J. H., Mattson, M. P., and Krieglstein, J. (2002). Nerve growth factor survival signaling in cultured hippocampal neurons is mediated through TrkA and requires the common neurotrophin receptor P75. Neuroscience 115, 1089-1108.

Culmsee, C., Stumm, R. K., Schafer, M. K., Weihe, E., and Krieglstein, J. (1999). Clenbuterol induces growth factor mRNA, activates astrocytes, and protects rat brain tissue against ischemic damage. Eur J Pharmacol 379, 33-45.

Descamps, S., Pawlowski, V., Revillion, F., Hornez, L., Hebbar, M., Boilly, B., Hondermarck, H., and Peyrat, J. P. (2001). Expression of nerve growth factor receptors and their prognostic value in human breast cancer. Cancer Res 61, 4337-4340.

Ding, L., Huang, D., Wang, J., and Li, S. (2007). Determination of gambogic acid in human plasma by liquid chromatography-atmospheric pressure chemical ionization-mass spectrometry. J Chromatogr B Analyt Technol Biomed Life Sci 846, 112-118.

Dolle, L., Adriaenssens, E., El Yazidi-Belkoura, I., Le Bourhis, X., Nurcombe, V., and Hondermarck, H. (2004). Nerve growth factor receptors and signaling in breast cancer. Curr Cancer Drug Targets 4, 463-470.

Douma, S., Van Laar, T., Zevenhoven, J., Meuwissen, R., Van Garderen, E., and Peeper, D. S. (2004). Suppression of anoikis and induction of metastasis by the neurotrophic receptor TrkB. Nature 430, 1034-1039.

Ebadi, M., Bashir, R. M., Heidrick, M. L., Hamada, F. M., Refaey, H. E., Hamed, A., Helal, G., Baxi, M. D., Cerutis, D. R., and Lassi, N. K. (1997). Neurotrophins and their receptors in nerve injury and repair. Neurochem Int 30, 347-374.

Faherty, C. J., Xanthoudakis, S., and Smeyne, R. J. (1999). Caspase-3-dependent neuronal death in the hippocampus following kainic acid treatment. Brain Res Mol Brain Res 70, 159-163.

George, D. J., Dionne, C. A., Jani, J., Angeles, T., Murakata, C., Lamb, J., and Isaacs, J. T. (1999). Sustained in vivo regression of Dunning H rat prostate cancers treated with combinations of androgen ablation and Trk tyrosine kinase inhibitors, CEP-751 (KT-6587) or CEP-701 (KT-5555). Cancer Res 59, 2395-2401.

Guegan, C., Onteniente, B., Makiura, Y., Merad-Boudia, M., Ceballos-Picot, I., and Sola, B. (1998). Reduction of cortical infarction and impairment of apoptosis in NGF-transgenic mice subjected to permanent focal ischemia. Brain Res Mol Brain Res 55, 133-140.

Guo, Q., Qi, Q., You, Q., Gu, H., Zhao, L., and Wu, Z. (2006). Toxicological studies of gambogic acid and its potential targets in experimental animals. Basic Clin Pharmacol Toxicol 99, 178-184.

Guo, Q. L., You, Q. D., Wu, Z. Q., Yuan, S. T., and Zhao, L. (2004). General gambogic acids inhibited growth of human hepatoma SMMC-7721 cells in vitro and in nude mice. Acta Pharmacol Sin 25, 769-774.

Hughes, P. E., Alexi, T., Hefti, F., and Knusel, B. (1997). Axotomized septal cholinergic neurons rescued by nerve growth factor or neurotrophin-4/5 fail to express the inducible transcription factor c-Jun. Neuroscience 78, 1037-1049.

Jun, Y., Guo, Q. L., You, Q. D., Zhao, L., Gu, H. Y., Yang, Y., Zhang, H. W., Tan, Z., and Wang, X. (2006). Gambogic acid induced G2/M phase cell cycle arrest via disturbing CDK7 mediated phosphorylation of CDC2/P34 in human gastric carcinoma BGC-823 cells. Carcinogenesis.

Kaplan, D. R., and Stephens, R. M. (1994). Neurotrophin signal transduction by the Trk receptor. J Neurobiol 25, 1404-1417.

Kasibhatla, S., Jessen, K. A., Maliartchouk, S., Wang, J. Y., English, N. M., Drewe, J., Qiu, L., Archer, S. P., Ponce, A. E., Sirisoma, N., et al. (2005). A role for transferrin receptor in triggering apoptosis when targeted with gambogic acid. Proc Natl Acad Sci USA 102, 12095-12100.

Kokaia, Z., Andsberg, G., Martinez-Serrano, A., and Lindvall, O. (1998). Focal cerebral ischemia in rats induces expression of P75 neurotrophin receptor in resistant striatal cholinergic neurons. Neuroscience 84, 1113-1125.

Kume, T., Nishikawa, H., Tomioka, H., Katsuki, H., Akaike, A., Kaneko, S., Maeda, T., Kihara, T., and Shimohama, S. (2000). p75-mediated neuroprotection by NGF against glutamate cytotoxicity in cortical cultures. Brain Res 852, 279-289.

Lee, F. S., and Chao, M. V. (2001). Activation of Trk neurotrophin receptors in the absence of neurotrophins Proc Natl Acad Sci USA 98, 3555-3560.

Lee, T. H., Kato, H., Chen, S. T., Kogure, K., and Itoyama, Y. (1998). Expression of nerve growth factor and trkA after transient focal cerebral ischemia in rats. Stroke 29, 1687-1696; discussion 1697.

LeSauteur, L., Wei, L., Gibbs, B. F., and Saragovi, H. U. (1995). Small peptide mimics of nerve growth factor bind TrkA receptors and affect biological responses. J Biol Chem 270, 6564-6569.

Lindsay, R. M. (1996). Role of neurotrophins and trk receptors in the development and maintenance of sensory neurons: an overview. Philos Trans R Soc Lond B Biol Sci 351, 365-373.

Lippa, B., Morris, J., Corbett, M., Kwan, T. A., Noe, M. C., Snow, S. L., Gant, T. G., Mangiaracina, M., Coffey, H. A., Foster, B., et al. (2006). Discovery of novel isothiazole inhibitors of the TrkA kinase: structure-activity relationship, computer modeling, optimization, and identification of highly potent antagonists. Bioorg Med Chem Lett 16, 3444-3448.

Liu, W., Guo, Q. L., You, Q. D., Zhao, L., Gu, H. Y., and Yuan, S. T. (2005). Anticancer effect and apoptosis induction of gambogic acid in human gastric cancer line BGC-823. World J Gastroenterol 11, 3655-3659.

Luk, Y. O., Chen, W. Y., Wong, W. J., Hu, H. H., Hsu, L. C., Chern, C. M., Huang, K. J., and Law, S. L. (2004). Treatment of focal cerebral ischemia with liposomal nerve growth factor. Drug Deliv 11, 319-324.

Marshall, J. L., Kindler, H., Deeken, J., Bhargava, P., Vogelzang, N. J., Rizvi, N., Luhtala, T., Boylan, S., Dordal, M., Robertson, P., et al. (2005). Phase I trial of orally administered CEP-701, a novel neurotrophin receptor-linked tyrosine kinase inhibitor. Invest New Drugs 23, 31-37.

McArthur, J. C., Yiannoutsos, C., Simpson, D. M., Adornato, B. T., Singer, E. J., Hollander, H., Marra, C., Rubin, M., Cohen, B. A., Tucker, T., et al. (2000). A phase II trial of nerve growth factor for sensory neuropathy associated with HIV infection. AIDS Clinical Trials Group Team 291. Neurology 54, 1080-1088.

McGregor, L. M., McCune, B. K., Graff, J. R., McDowell, P. R., Romans, K. E., Yancopoulos, G. D., Ball, D. W., Baylin, S. B., and Nelkin, B. D. (1999). Roles of trk family neurotrophin receptors in medullary thyroid carcinoma development and progression. Proc Natl Acad Sci USA 96, 4540-4545.

McMahon, S. B., and Priestley, J. V. (1995). Peripheral neuropathies and neurotrophic factors: animal models and clinical perspectives. Curr Opin Neurobiol 5, 616-624.

Mufson, E. J., Lavine, N., Jaffar, S., Kordower, J. H., Quirion, R., and Saragovi, H. U. (1997). Reduction in p140-TrkA receptor protein within the nucleus basalis and cortex in Alzheimer's disease. Exp Neurol 146, 91-103.

Nadler, J. V., Perry, B. W., Gentry, C., and Cotman, C. W. (1980). Degeneration of hippocampal CA3 pyramidal cells induced by intraventricular kainic acid. J Comp Neurol 192, 333-359.

Olson, L. (1993). NGF and the treatment of Alzheimer's disease. Exp Neurol 124, 5-15.

Saragovi, H. U., Fitzpatrick, D., Raktabutr, A., Nakanishi, H., Kahn, M., and Greene, M. I. (1991). Design and synthesis of a mimetic from an antibody complementarity-determining region. Science 253, 792-795.

Saragovi, H. U., Greene, M. I., Chrusciel, R. A., and Kahn, M. (1992). Loops and secondary structure mimetics: development and applications in basic science and rational drug design. Biotechnology (NY) 10, 773-778.

Sayeed, I., Guo, Q., Hoffman, S. W., and Stein, D. G. (2006). Allopregnanolone, a progesterone metabolite, is more effective than progesterone in reducing cortical infarct volume after transient middle cerebral artery occlusion. Ann Emerg Med 47, 381-389.

Schauwecker, P. E., and Steward, O. (1997). Genetic determinants of susceptibility to excitotoxic cell death: implications for gene targeting approaches. Proc Natl Acad Sci USA 94, 4103-4108.

Sperk, G., Lassmann, H., Baran, H., Kish, S. J., Seitelberger, F., and Hornykiewicz, O. (1983). Kainic acid induced seizures: neurochemical and histopathological changes. Neuroscience 10, 1301-1315.

Tabakman, R., Jiang, H., Shahar, I., Arien-Zakay, H., Levine, R. A., and Lazarovici, P. (2005). Neuroprotection by NGF in the PC12 in vitro OGD model: involvement of mitogen-activated protein kinases and gene expression. Ann NY Acad Sci 1053, 84-96.

Urfer, R., Tsoulfas, P., O'Connell, L., Shelton, D. L., Parada, L. F., and Presta, L. G. (1995). An immunoglobulin-like domain determines the specificity of neurotrophin receptors. Embo J 14, 2795-2805.

Verrall, M. (1994). Lay-offs follow suspension of clinical trials of protein. Nature 370, 6.

Weeraratna, A. T., Arnold, J. T., George, D. J., DeMarzo, A., and Isaacs, J. T. (2000). Rational basis for Trk inhibition therapy for prostate cancer. Prostate 45, 140-148.

Wu, Z. Q., Guo, Q. L., You, Q. D., Zhao, L., and Gu, H. Y. (2004). Gambogic acid inhibits proliferation of human lung carcinoma SPC-A1 cells in vivo and in vitro and represses telomerase activity and telomerase reverse transcriptase mRNA expression in the cells. Biol Pharm Bull 27, 1769-1774.

Zhang, H. Z., Kasibhatla, S., Wang, Y., Herich, J., Guastella, J., Tseng, B., Drewe, J., and Cai, S. X. (2004). Discovery, characterization and SAR of gambogic acid as a potent apoptosis inducer by a HTS assay. Bioorg Med Chem 12, 309-317.

Zhang, Y., Dang, C., Ma, Q., and Shimahara, Y. (2005). Expression of nerve growth factor receptors and their prognostic value in human pancreatic cancer. Oncol Rep 14, 161-171.

Zhang, Y., Tatsuno, T., Carney, J. M., and Mattson, M. P. (1993). Basic FGF, NGF, and IGFs protect hippocampal and cortical neurons against iron-induced degeneration. J Cereb Blood Flow Metab 13, 378-388.

Zhao, L., Guo, Q. L., You, Q. D., Wu, Z. Q., and Gu, H. Y. (2004). Gambogic acid induces apoptosis and regulates expressions of Bax and Bcl-2 protein in human gastric carcinoma MGC-803 cells. Biol Pharm Bull 27, 998-1003.

Having now fully described this invention, it will be understood by those of ordinary skill in the art that the same can be performed within a wide and equivalent range of conditions, formulations and other parameters without affecting the scope of the invention or any embodiment thereof. All patents, patent applications and publications cited herein are fully incorporated by reference herein in their entirety.

1. An amine analogue of gambogic acid, where the amine group has the formula (CH₂NR₁R₂), where R₁ and R₂ are, individually, hydrogen, optionally substituted lower alkyl, optionally substituted aryl, optionally substituted lower aralkyl, or optionally substituted lower alkylaryl groups,
where the substituents are one or more substituents selected from the group consisting of halo, hydroxy, carboxyl, alkoxycarbonyl, amino, nitro, cyano, C₁-C₆ acylamino, C₁-C₆ aminoacyl, C₁-C₆ acyloxy, C₁-C₆ alkoxy, aryloxy, alkylthio, C₆-C₁O aryl, C_{d}-C₇ cycloalkyl, C₂-C₆ alkenyl, C₂-C₆ alkynyl, C₆-C₁O aryl(C₂-C₆)alkenyl, C₆-C₁O aryl(C₂-C₆)alkynyl, saturated or partially saturated 5-7 membered heterocyclo group, or heteroaryl,
or a pharmaceutically acceptable salt or prodrug thereof.

2. An amine analogue of gambogic acid, where the amide group has the formula (CONH₂), or a pharmaceutically acceptable salt or prodrug thereof.

3. A pharmaceutical composition comprising the compound of claim 1, and a pharmaceutically acceptable carrier.

4. The pharmaceutical composition of claim 3, further comprising a compound other than an amine analogue of gambogic acid, which compound has neuroprotective activity, myelin protecting activity, or modulates the release of a neurotransmitter.

5. A method of treating or preventing a neurodegenerative disorder responsive to the agonism of TrkA in an mammal, comprising administering to a mammal in need of such treatment an effective amount of a compound of claim 1.

6. The method of claim 5, wherein the disorder is stroke, Parkinson's Disease, mild cognitive impairment, Alzheimer' s disease, epilepsy, ALS, and other neurodegenerative diseases.

7. A method of providing neuroprotection either before, during, or after an ischemic event, comprising administering a compound of claim 1, to a patient in need of neuroprotection.

8. The method of claim 7, wherein the compound or composition is administered following a stroke or other ischemic event.

9. The method of claim 7, wherein the compound or composition is administered following exposure to a neurotoxin.

10. The method according to claim 7, wherein said compound or composition is administered together with at least one known neuroprotective agent, or a pharmaceutically acceptable salt of the agent.

11. A method of treating or preventing a demyelinating disorder, comprising administering a compound of claim 1, to a patient in need of treatment or prevention thereof.

12. The method of claim 11, wherein the demyelinating disorder is multiple sclerosis.

13. The method of claim 11, wherein the compound or composition is administered together with interferon or a pegylated version thereof.
